(19) United States
(12) Patent Application Publication
(10) Pub. No.: US 2025/0171851 A1
(43) Pub. Date: May 29, 2025

(54) BIOMARKER miR-32533 FOR COGNITIVE IMPAIRMENT-RELATED DISEASE AND USE THEREOF

(71) Applicant: INSTITUTE OF MEDICINAL BIOTECHNOLOGY, CAMS & PUMC, BEIJING (CN)

(72) Inventors: RUI LIU, BEIJING (CN); ZHUORONG LI, BEIJING (CN); LI ZENG, BEIJING (CN); KAIYUE ZHAO, BEIJING (CN); ZHONGDI CAI, BEIJING (CN)

(73) Assignee: INSTITUTE OF MEDICINAL BIOTECHNOLOGY, CAMS & PUMC, BEIJING (CN)

(21) Appl. No.: 19/051,528

(22) Filed: Feb. 12, 2025

Related U.S. Application Data
(63) Continuation-in-part of application No. PCT/CN2023/111859, filed on Aug. 9, 2023.

(30) Foreign Application Priority Data
Aug. 17, 2022 (CN) 202210983855.5

(51) Int. Cl. C12Q 1/6883; C12Q 1/6851

(52) U.S. Cl. CPC C12Q 1/6883; C12Q 1/6851; C12Q 2600/178

(57) ABSTRACT

Disclosed is a biomarker miR-32533 for cognitive impairment-related diseases and use thereof. The miR-32533 includes the nucleotide sequence set forth in SEQ ID NO: 1, and can be used as a biomarker for detecting the cognitive impairment-related diseases, especially Alzheimer's disease (AD). The miR-32533 can specifically bind to CREB5 gene, reduce an expression level of CREB5 gene, and inhibit generation of extracellular β-amyloid (Aβ), thereby effectively preventing and treating AD.

### CROSS REFERENCE TO RELATED APPLICATION

The present application is a continuation-in-part of International Patent Application No. PCT/CN2023/111859, filed on Aug. 9, 2023, which claims priority to the Chinese Patent Application No. CN202210983855.5, filed with the China National Intellectual Property Administration (CNIPA) on Aug. 17, 2022, and entitled “BIOMARKER miR-32533 FOR COGNITIVE IMPAIRMENT-RELATED DISEASE AND USE THEREOF”, which is incorporated herein by reference in its entirety.

### REFERENCE TO SEQUENCE LISTING

A computer readable XML file entitled “Sequence Listing”, that was created on December 20, with a file size of 15,852 bytes, contains the sequence listing for this application, has been filed with this application, and is hereby incorporated by reference in its entirety.

### TECHNICAL FIELD

The present disclosure belongs to the technical field of biological detection, and specifically relates to a biomarker miR-32533 for cognitive impairment-related diseases and use thereof.

### BACKGROUND

As the population ages globally, the incidence and mortality of cognitive impairment-related diseases and senile dementia are on the rise year by year, posing a serious threat to the physical and mental health of the elderly, and imposing a heavy burden on families and society. The cognitive impairment-related diseases include Alzheimer's disease (AD), Parkinson's disease, Huntington's disease, and vascular dementia. Among them, AD is the most common type of senile dementia. It is also a progressive neurodegenerative disease with insidious onset and high correlation with age. Clinically, it is characterized by a decline in cognitive function, particularly in learning and memory. The main pathological mechanisms of AD involve the extracellular aggregation of β-amyloid (Aβ) to form senile plaques, intracellular hyperphosphorylation of microtubule-associated Tau protein leading to neurofibrillary tangles (NFTs), neuroinflammation, and synaptic dysfunction, which ultimately lead to neuronal death or loss. Currently, anti-AD drugs approved by the Food and Drug Administration (FDA) can only alleviate mild and moderate AD symptoms, albeit with the presence of varying levels of adverse reactions. As of now, there is no drug that can reverse the pathological process of AD to achieve a cure. At this stage, research on novel drug targets and specific biodiagnostic markers is of great value for effectively treating AD, assessing disease progression, and improving the quality of life for patients.

Non-coding RNAs (ncRNAs) are abundantly expressed in the central nervous system and play an important role in epigenetic regulation of Aβ production, Tau protein phosphorylation, and endogenous apoptosis signaling pathways. Within the spectrum of cognitive impairment-related diseases, there is a need for further exploration and research on novel ncRNAs with brain tissue specificity. Meanwhile, the spatiotemporal characteristics, pathogenesis pathways, and biological functions of ncRNAs throughout the pathological process of AD have not been systematically studied. Therefore, it is of great significance for understanding the early molecular events of AD, proposing molecular mechanisms at the epidemiological level, and drug discovery strategies by discovering novel ncRNA molecules and elucidating the biological functions of abnormal ncRNAs in AD.

### SUMMARY

An objective of the present disclosure is to provide a biomarker miR-32533 for cognitive impairment-related diseases and use thereof. The biomarker can be used to design early detection kits, effectively treat AD, assess disease progression, and improve the life quality of patients.

The present disclosure provides a biomarker miR-32533 for cognitive impairment-related diseases, where the nucleotide sequence of the biomarker miR-32533 is set forth in SEQ ID NO: 1.

The present disclosure further provides use of the biomarker miR-32533 in preparation of a drug for treating cognitive impairment-related diseases.

In some embodiments, the cognitive impairment-related diseases include AD.

In some embodiments, treating cognitive impairment-related diseases includes one or more of the following I) to VI):
I) increasing an expression level of ADAM10 in a non-amyloid metabolic pathway of Aβ;
II) reducing expression levels of BACE1 and PS1 in an amyloid metabolic pathway of the Aβ;
III) inhibiting generation of the Aβ;
IV) exerting a neuroprotective effect;
V) increasing a cell viability and reducing apoptosis; and
VI) reducing an expression level of CREB5.

The present disclosure further provides a drug for treating cognitive impairment-related diseases, where active ingredients of the drug include miR-32533 and/or a substance that promote expression of the miR-32533.

In some embodiments, the substance includes chemical small-molecule drugs, nucleic acid drugs, and antibody drugs.

The present disclosure further provides use of the miR-32533 in preparation of a kit being one or more selected from the group consisting of a) to d):
a) a kit for screening a population with the cognitive impairment-related diseases;
b) a kit for diagnosing the population with the cognitive impairment-related diseases;
c) a kit for monitoring a treatment status of the population with the cognitive impairment-related diseases; and
d) a kit for monitoring prognosis of the population with the cognitive impairment-related diseases.

In some embodiments, the cognitive impairment-related diseases include AD.

The present disclosure further provides a kit for screening cognitive impairment-related diseases, including a reagent for detecting the miR-32533 and/or a reagent for detecting an expression level of CREB5.

The present disclosure further provides use of CREB5 gene as a target in preparation of a drug for treating cognitive impairment-related diseases.

In some embodiments, the cognitive impairment-related diseases include AD.

### Beneficial Effects:

In the present disclosure, the biomarker miR-32533, with the nucleotide sequence set forth in SEQ ID NO: 1, is characterized by high stability and resistance to degradation by endogenous RNA enzymes. It is significantly downregulated in AD model cells, AD model animals, and AD patient plasma, and therefore may be used as a biomarker for detecting AD. In addition, flow cytometry, MTS colorimetry (colorimetric assay by 3-(4,5-dimethylthiazol-2-yl)-5-(3-carboxymethoxyphenyl) pentane-2,4-dione disodium salt), Western blotting, gene overexpression, and gene silencing are conducted in the present disclosure to explore the spatiotemporal distribution, pathological effects, and molecular mechanisms of miR-32533 in AD. It is elucidated that miR-32533 can improve cell viability and reduce apoptosis. At the same time, the miR-32533 can promote the expression level of ADAM10 in the non-amyloid metabolic pathway of Aβ, reduce the expression levels of BACE1 and PS1 in the amyloid metabolic pathway of Aβ, and inhibit the generation of Aβ, thus exerting a neuroprotective effect. Moreover, bioinformatics analysis is conducted using online databases (TargetScan, miRDB, and Tarbase) to further explore the pharmacodynamic effects and mechanisms of miR-32533 in neuroprotective effect during the pathological process of AD. The results show that the miR-32533 specifically binds to CREB5 and specifically negatively regulates the expression level of CREB5 gene at both the transcriptional and translational levels. Therefore, promoting the expression of miR-32533, reducing the expression level of CREB5, and inhibiting the generation of Aβ can effectively prevent and treat AD.

### BRIEF DESCRIPTION OF THE DRAWINGS

To describe the technical solutions in the examples of the present disclosure or in the prior art more clearly, the accompanying drawings required for the examples will be briefly described below.

FIG. 1 is a heat map showing the expression level of miR-32533 in the cerebral cortex of APP/PS1 mice and WT wild-type control mice detected by RNA high-throughput sequencing.

FIG. 2 shows the expression level of miR-32533 detected by RT-PCR in APP/PS1 mice and WT wild-type control mice.

FIG. 3 shows the results of the ROC curve analysis of the diagnostic predictive value of miR-32533 in APP/PS1 mice.

FIG. 4 shows the expression level of miR-32533 detected by RT-PCR in 5×FAD mice.

FIG. 5 shows the expression level of miR-32533 in APPswe cells at different time points after copper ion treatment detected by RT-PCR.

FIG. 6 is the results of MTS assay showing the effect of miR-32533 on the cell viability of APPswe cells induced by copper ions.

FIG. 7 shows the quantitative results of the miR-32533 on copper ion-induced APPswe cell apoptosis detected by flow cytometry.

FIG. 8 shows representative images of flow cytometry analysis of the effect of the miR-32533 on copper ion-induced APPswe cell apoptosis.

FIG. 9 shows representative Western blotting images of ADAM10, BACE1, PS1, APP, Aβ₁₋₄₂, and Bax in APPswe cells after transfection with miR-32533, miR-32533 inhibitor, and negative sequence control (NC/NCI).

FIG. 10 shows the representative protein relative expression levels of ADAM10, BACE1, PS1, APP, Aβ₁₋₄₂, and Bax in APPswe cells after transfection with miR-32533, miR-32533 inhibitor, and negative sequence control (NC/NCI).

FIG. 11 shows the expression level changes of miR-32533 in the plasma of AD patients and age-matched healthy adult volunteers (HAV) (n=10, HAV; n=11, AD patients).

FIG. 12 shows the correlation between the expression level of miR-32533 and MMSE score.

FIG. 13 shows the ROC curve analysis for the diagnostic predictive value of miR-32533 in AD patients and HAV controls.

FIG. 14 shows the expression levels of CREB5 in the cortex of APP/PS1 mice at 1, 3, 6, and 9 months of age detected by RT-PCR.

FIG. 15 shows the expression level of CREB5 in the cortex of 5×FAD mice detected by RT-PCR.

FIG. 16 shows the correlation between the miR-32533 and CREB5 expression level in the cerebral cortex of APP/PS1 mice.

FIG. 17 shows the expression of CREB5 mRNA in APPswe cells after transfection with miR-32533 mimics and miR-32533 inhibitor.

FIG. 18 shows representative images of the effect of miR-32533 overexpression and miR-32533 silencing on CREB5 protein expression level in APPswe cells.

FIG. 19 shows quantitative results of CREB5 protein expression level in APPswe cells with miR-32533 overexpression and miR-32533 silencing.

FIG. 20 shows a schematic diagram of the construction of the dual luciferase reporter gene plasmids with wild-type pmirGLO-CREB5-3′UTR-WT and mutant pmirGLO-CREB5-3′UTR-MUT.

FIG. 21 shows the effect of co-transfection of the miR-32533 (or negative sequence control NC) and pmirGLO-CREB5-3′UTR-WT (or pmirGLO-CREB5-3′UTR-MUT) on a luciferase activity of HEK293 cells detected by dual luciferase reporter gene assay.

### DETAILED DESCRIPTION OF THE EMBODIMENTS

The present disclosure provides a biomarker miR-32533 for cognitive impairment-related diseases, the nucleotide sequence of which is set forth in SEQ ID NO: 1, specifically: 5′-UCUCUUCUGCUCUGUGUCACAGC-3′. In the present disclosure, the nucleotide sequence set forth in SEQ ID NO: 1 is the nucleotide sequence of the mature form of the miR-32533, and the precursor of the miR-32533 has a nucleotide sequence preferably set forth in SEQ ID NO: 2, specifically: 5′-UCUCUUCUGCUCUGUGUCACAGCAACAGAAAGCUGCACUGCUGAGGAACA-3′.

In the present disclosure, high-throughput sequencing is conducted to determine the expression profiles of ncRNAs in the brains of APP/PS1 mice at 1, 3, 6, and 9 months of age, and miR-32533 with clear characteristic changes is selected and excavated, which shows high stability, and can resist degradation by endogenous RNases. The expression of miR-32533, which is tissue-specific, disease-specific, and highly species-conserved in the pathological process of AD, is verified at multiple levels in vivo and in vitro. The miRNA real-time fluorescence quantitative PCR (RT-PCR) has confirmed that miR-32533 is significantly downregulated in AD model cells, AD model animals, and the plasma of AD patients. Moreover, the receiver operating characteristic (ROC) curve analysis has showed that miR-32533 can exhibit desirable clinical diagnostic value as a biomarker in the plasma of AD patients and healthy controls. As a result, the miR-32533 is associated with AD and can be used as a biomarker for AD diagnosis.

The present disclosure further provides use of the miR-32533 in preparation of a kit being one or more selected from the group consisting of a) to d):
a) a kit for screening a population with the cognitive impairment-related diseases;
b) a kit for diagnosing the population with the cognitive impairment-related diseases;
c) a kit for monitoring a treatment status of the population with the cognitive impairment-related diseases; and
d) a kit for monitoring prognosis of the population with the cognitive impairment-related diseases.

In the present disclosure, miR-32533 with a novel sequence structure is discovered for the first time, and its expression profile and biological functions during the pathological process of cognitive impairment-related diseases are systematically explained. Based on the fact that the expression level of miR-32533 can reflect whether cognitive impairment-related diseases occur, miR-32533 is used as a biomarker to determine whether cognitive impairment-related diseases occur by detecting the relative expression level of miR-32533, providing a new diagnostic approach for diagnosing the cognitive impairment-related diseases. In addition, the miR-32533 can be used as a biomarker to screen population suffering from cognitive impairment-related diseases, as well as to monitor the treatment status and prognosis of patients, determining the outcome of cognitive impairment-related diseases, and thus improving the life quality of patients. The cognitive impairment-related diseases include AD.

The present disclosure further provides a kit for screening AD, where the kit detects the biomarker miR-32533. In the present disclosure, the biomarker miR-32533 as a detection target provides a new idea for diagnosing the cognitive impairment-related diseases, which is of great value in determining the prognosis of AD disease and improving the life quality of patients. The cognitive impairment-related diseases preferably include AD.

The present disclosure further provides use of the biomarker miR-32533 in preparation of a drug for treating the cognitive impairment-related diseases.

In the present disclosure, the cognitive impairment-related diseases preferably include AD.

In the present disclosure, beyond reflecting the occurrence of cognitive impairment-related disease through the expression level of miR-32533, it can also be used as a molecular target to treat the cognitive impairment-related diseases. High-throughput sequencing analysis of APP/PS1 mice and control mice has revealed that the expression level of CREB5 during the pathological process of AD is negatively correlated with the expression of miR-32533 in APP/PS1 mice, and discovered that CREB5 is associated with the disease for the first time. Bioinformatics software is used to predict and analyze a potential binding site of a 3′ untranslated region (3′ UTR) in CREB5 with the miR-32533, and a dual luciferase reporter gene is used to detect the specific binding of miR-32533 to CREB5. The results show that the biomarker miR-32533 can specifically negatively regulate the expression level of CREB5 at both the transcriptional and translational levels. At the same time, gene intervention is conducted in

APPswe cells to further explore the molecular mechanism of miR-32533/CREB5 signaling axis involved in AD. Western blot analysis reveals that the miR-32533 improves the pathological manifestations of AD by promoting the expression level of ADAM10 in the non-amyloid metabolic pathway of Aβ, reducing the expression levels of BACE1 and PSI in the amyloid metabolic pathway of Aβ, inhibiting the generation of Aβ, and alleviating Aβ-induced neurotoxicity. Accordingly, promoting the expression level of miR-32533, reducing the expression level of CREB5, and inhibiting the generation of Aβ can achieve the treatment of AD.

The present disclosure further provides a drug for treating AD, where an active ingredient of the drug includes miR-32533 and/or substances that promote expression of the miR-32533.

In the present disclosure, the substances that promote expression of the miR-32533 preferably include a chemical small-molecule drug, a nucleic acid drug, and an antibody drug. The drug directly uses miR-32533 as an active ingredient or uses substances that promote the expression of miR-32533 as an active ingredient, and can promote the expression level of miR-32533, reduce the expression level of CREB5, inhibit the generation of Aβ, so as to achieve the purpose of treating AD. There is no strict requirement on the source of the substances that promote the expression of miR-32533. Any substance that can promote the expression of miR-32533, such as chemical small-molecule drugs, nucleic acid molecules, and antibody molecules, falls within the protection scope of the present disclosure.

In order to further illustrate the present disclosure, the biomarker miR-32533 for cognitive impairment-related diseases and the use thereof provided in the present disclosure are described in detail below in connection with accompanying drawings and examples, but they should not be understood as limiting the claimed scope of the present disclosure.

In the specific examples of the present disclosure, unless otherwise specified, steps involved all are routine steps, and reagents used can be conventionally purchased or prepared according to product instructions.

### EXAMPLE 1

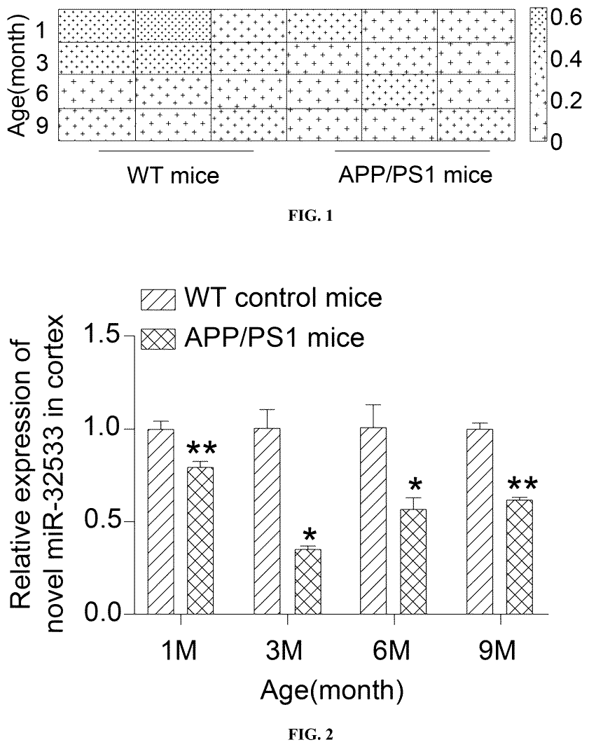

1. Amyloid precursor protein/presenilin 1 mice (referred to as APP/PS1 mice, purchased from Zhishan (Beijing) Health Medical Research Institute Co., Ltd. (Laboratory Animal Production License No.: SCXK (Beijing) 2021-0010)) and wild-type mice (referred to as WT mice) were used as experimental materials. The cerebral cortex of APP/PS1 mice and wild-type mice aged 1, 3, 6, and 9 months were taken separately, and the expression profiles of ncRNAs in the cerebral cortex of different mice at different ages were detected by high-throughput sequencing. The results showed that there was a differentially expressed ncRNA in the cerebral cortex of APP/PS1 mice and wild-type mice of the same age (as shown in FIG. 1), referred to as miR-32533. The nucleotide sequence was determined shown in SEQ ID NO: 1 (mature form), specifically, 5′-UCUCUUCUGCUCUGUGUCACAGC-3′; the precursor sequence of miR-32533 (referred to as Pre-miR-32533) was shown in SEQ ID NO: 2, specifically: 5′-UCUCUUCUGCUCUGUGUCACAGCAACAGAAAGCUGCACUGCUGAGGAACA-3′. The above steps were entrusted to Sangon Biotech (Shanghai) Co., Ltd.

### EXAMPLE 2

### Expression Changes of the miR-32533 in AD Model Animals

### 1. RT-PCR Detection

APP/PS1 mice at 1, 3, 6, and 9 months of age (referred to as APP/PS1 mice, purchased from Zhishan (Beijing) Health Medical Research Institute Co., Ltd.) and wild-type control mice at 1, 3, 6, and 9 months of age (referred to as WT control mice) were used as experimental materials. RT-PCR was conducted to detect the expression levels of miR-32533 in different mice at different ages, including the following steps:

### 1.1 Total RNA Extraction

An Ultrapure RNA extraction kit of Jiangsu CWBIO Biotech Co., Ltd. was used, and steps were as follows:
(1) The above animal tissues were cut into small pieces and put into EP tubes, where 1 mL of TRIzol Reagent was added for every 30 mg of tissues, obtained samples were repeatedly pipetted and beat several times to fully lyse the samples, allowed to stand at room temperature for 5 min to completely separate the protein-nucleic acid complex, and then homogenized in a homogenizer.
(2) 200 μL of chloroform was added to the homogenized tissue obtained in step (1) at a ratio of 200 μL of chloroform per 1 mL of TRIzol, the tube was covered and shaken vigorously for 15 s, placed at room temperature for 2 min and centrifuged at 4° C., 12,000 rpm for 10 min. At this time, the sample was divided into 3 layers: the lower red organic phase, the middle layer, and the upper colorless aqueous phase, and the upper aqueous phase was transferred to a new RNase-Free centrifuge tube.
3) An equal volume of ethanol prepared with 70% RNase-free water was added to the aqueous phase solution, inverted to mix well, a mixed solution was added to an adsorption column in the collection tube, centrifuged at 12,000 rpm for 20 s, waste liquid in the collection tube was discarded, and the adsorption column was put back into the collection tube.
(4) 700 μL of a Buffer RW1 was added to the adsorption column, centrifuged at 12,000 rpm for 20 s, and the waste liquid in the collection tube was discarded.
(5) 500 μL of a Buffer RW2 was added to the adsorption column, centrifuged at 12,000 rpm for 20 s, the waste liquid in the collection tube was discarded, and the adsorption column was put back into the collection tube; and this step was repeated once.
(6) The adsorption column was placed back into the collection tube of step (5), centrifuged at 12,000 rpm for 2 min, the waste liquid was discarded, and the adsorption column was placed at room temperature for several minutes to completely dry.
(7) The adsorption column was placed in a new RNase-free centrifuge tube, and 30 μL of RNase-free water was added to a middle part of the adsorption column. The column was placed at room temperature for 1 min and centrifuged at 12,000 rpm for 1 min. An RNA solution was collected, and the concentration and purity of the RNA were determined using a quartz plate on a Spark 20M multifunctional microplate reader. The RNA was stored at −80° C. to prevent degradation.
   1.2 RT-PCR of miRNA

Reverse transcription of miRNA was conducted using the miRNA 1st Strand cDNA Synthesis Kit, and qPCR experiments were conducted using the miRNA Universal SYBR qPCR Master Mix Kit, so as to detect the expression level of miR-32533 in tissues of APP/PS1 mice at 1, 3, 6, and 9 months of age and wild-type control mice at 1, 3, 6, and 9 months of age, including the following steps:

1.2.1 Reverse Transcription of miRNA
(1) Genomic DNA removal reaction: all reagents were equilibrated to room temperature for later use. A 20 μL reaction system was prepared according to Table 1 and incubated at 42° C. for 2 min:

TABLE 1
Reaction system for genomic DNA removal

| Reagent component | Volume |
|---|---|
| RNase-free ddH₂O | Up to 10 μL |
| 5× gDNA wiper Mix | 2 μL |
| RNA sample | 1 μg |

(2) The first-strand cDNA synthesis was conducted, where a reaction system was shown in Table 2:

TABLE 2
Reaction system for first-strand cDNA synthesis

| Reagent component | Volume |
|---|---|
| RNase-free ddH₂O | Up to 20 μL |
| Mixed solution obtained in the previous step | 10 μL |
| Stem-loop reverse transcription primer (2 μM) | 1 μL |
| 10× RT Mix | 2 μL |
| HiScript II Enzyme Mix | 2 μL |

The nucleotide sequence of the stem-loop reverse transcription primer was set forth in SEQ ID NO: 3, specifically: 5′-GTCGTATCCAGTGCAGGGTCCGAGGTATTCGCACTGGATACGACGCTGTG-3′;

a program was set in the PCR amplification instrument as follows: 25° C., 5 min; 50° C., 15 min; 85° C., 5 min; the obtained cDNA product was stored at −20° C. or immediately subjected to PCR reaction.

### 1.2.2 RT-qPCR

20 ML of the qPCR reaction system was prepared according to Table 3, and 3 replicate wells were set up for each sample;

TABLE 3
qPCR reaction system

| | Reaction |
|---|---|
| Reagent component | volume |
| 2× miRNA Universal SYBR qPCR Master Mix | 10 μL |
| Forward primer (10 μM) | 0.4 μL |
| Reverse primer (10 μM) | 0.4 μL |
| mQ Primer R (10 μM) | 0.4 μL |
| Template cDNA | 2 μL |
| ddH₂O | 6.8 μL |

the nucleotide sequence of the forward primer was set forth in SEQ ID NO: 4, specifically: 5′-CGCGTCTCTTCTGCTCTGTGT-3′; the nucleotide sequence of the reverse primer was set forth in SEQ ID NO: 5, specifically: 5′-AGTGCAGGGTCCGAGGTATT-3′.

A program was set in the RT-qPCR instrument for qPCR reaction: initial denaturation: 95° C., 5 min; cycle reaction: 95° C., 10 s, 60° C., 30 s, 95° C., 15 s, cycle number 40; melting curve: 60° C., 60 s; 95° C., 15 s. The relative expression level of miRNA was calculated by a 2-AACT method with U6 as an internal reference to detect the relative expression level of miR-32533 in APP/PS1 mice and WT wild-type control mice. The nucleotide sequence of the stem-loop reverse transcription primer of U6 was set forth in SEQ ID NO: 6, specifically: 5′-GTCGTATCCAGTGCAGGGTCCGAGGTATTCGCACTGGATACGACAAAATA-3′; the nucleotide sequence of the forward primer for amplifying U6 was set forth in SEQ ID NO: 7, specifically: 5′-CAAATTCGTGAAGCGTTCCA-3′; the nucleotide sequence of the reverse primer for amplifying U6 was set forth in SEQ ID NO: 8, specifically: 5′-AGTGCAGGGTCCGAGGTATT-3′.

The results were shown in FIG. 2, where the results were expressed as mean±SEM (n=3), * indicated P<0.05 in APP/PS1 mice compared with WT control mice; ** indicated P<0.01 in APP/PS1 mice compared with WT control mice; M indicated month.

As shown in FIG. 2, there was a significant difference in the expression level of miR-32533 between APP/PS1 mice and WT wild-type control mice of the same age, and the miR-32533 was significantly downregulated in the cortex of APP/PS1 mice.

### 2. ROC Curve Detection

ROC curve analysis was conducted using GraphPad Prism Version 8.0 (GraphPad Prism Software, La Jolla, CA, USA). The sample values were back-substituted with the expression level of miR-32533 as an independent variable, and APP/PS1 mice and wild-type control (WT) mice of the same age as dependent variables. The ROC curves of each index were fitted with sensitivity as an ordinate (true positive rate) and 1-specificity as an abscissa (false positive rate). The value of biomarkers was determined in plasma for the independent diagnosis and combined diagnosis. The sensitivity and specificity were determined, the area under curve (AUC) was used as a comprehensive statistic for quantitative analysis to evaluate the diagnostic accuracy of diagnostic biomarkers. The closer the AUC was to 1, the better the diagnostic effect could be: i) AUC at 0.5 to 0.7 indicated a lower accuracy. ii) AUC at 0.7 to 0.9 indicated a certain degree of accuracy. iii) AUC above 0.9 indicated a high accuracy. iv) when AUC was 0.5, it indicated that the effect was ineffective and had no diagnostic value.

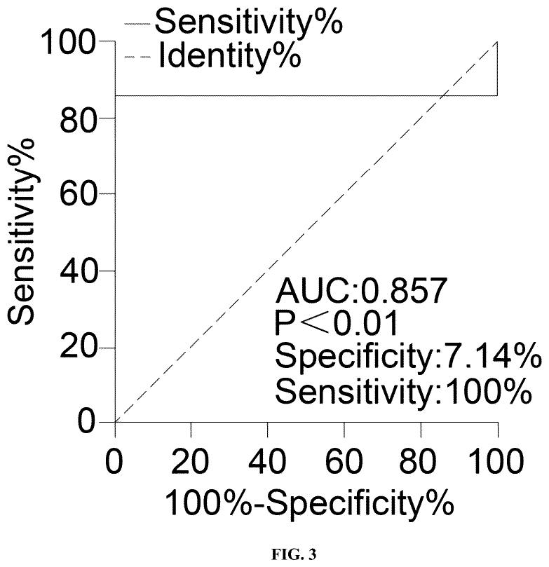

The results were shown in FIG. 3. According to FIG. 3, the miR-32533 showed a desirable diagnostic value as a biomarker for AD in APP/PS1 mice and WT control mice (AUC: 0.857, P<0.01 vs. WT), and could be used as a biomarker for diagnosing the AD.

### 3. Verification Experiment

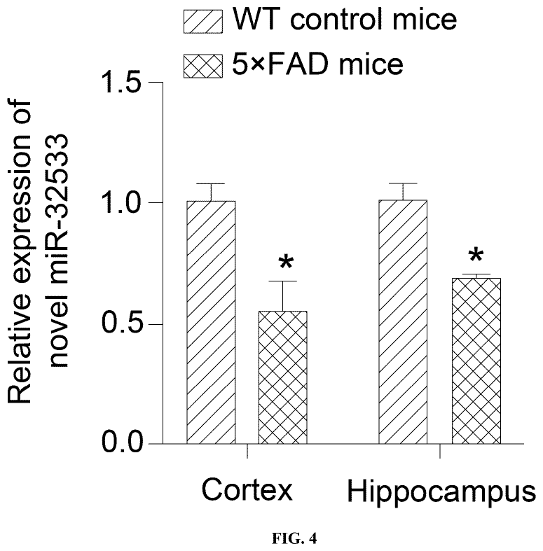

The cortex and hippocampus of 5× familiar AD mice (referred to as 5×FAD mice, kindly donated by the Academy of Military Science) and wild-type control mice (referred to as WT control mice) were used as experimental materials, and RT-PCR detection was conducted using the process in step 1 above. The test results were shown in FIG. 4, where the results were calculated as mean±SEM (n=3); * indicated P<0.05 of 5×FAD mice compared with WT control mice; ** indicated P<0.01 of 5×FAD mice compared with WT control mice; Cortex represented the cortex, and Hippocampus represented the hippocampus.

According to FIG. 4, miR-32533 was significantly downregulated in the cortex and hippocampus of 5×FAD mice. This further verified that the miR-32533 showed a desirable diagnostic value as the biomarker for AD.

### EXAMPLE 3

### Expression Changes of the miR-32533 in AD Model Cells

### 1. Cell Culture

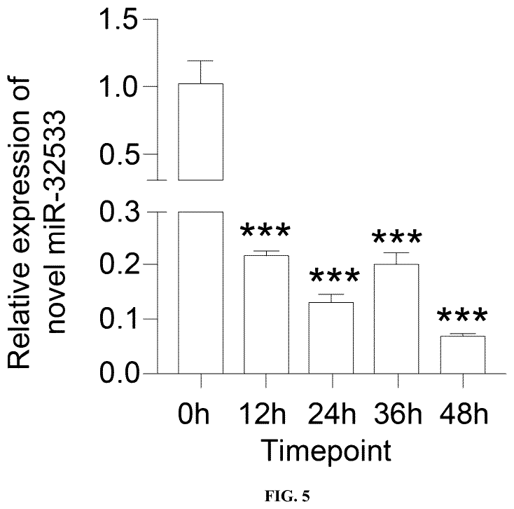

(1) APPswe cells were obtained by stably transfecting a Swedish human-mouse chimeric APP gene into human neuroblastoma SH-SY5Y cells, and the APPswe cells specifically produced excessive Aβ to induce neurotoxicity. The addition of copper ions induced overexpression of the endogenous APP gene, and at the same time formed a chelate with the overproduced Aβ to induce pathological changes such as Aβ toxicity, oxidative stress, and apoptosis, thereby simulating the AD pathological mechanism induced by exogenous factors and endogenous gene mutations.
(2) The APPswe cells in step (1) were cultured in a DMEM/F12 medium containing 2 mM L-glutamine, 400 μM G418, and 10% FBS. The APPswe cells were randomly divided into an experimental group and a control group, where the experimental group was treated with 300 μM copper ions, and the control group was not treated in any way. The expression level of miR-32533 in APPswe cells at different time points after copper ion treatment in the experimental group was randomly selected for detection. The results were shown in FIG. 5, where the results were expressed as mean±SEM (n=3), and *** indicated P<0.001 compared without copper ion addition.

According to FIG. 5, the miR-32533 was significantly downregulated in a time-dependent manner in APPswe cells treated with copper ions at different time points.
(3) The APPswe cells in step (1) were cultured in a DMEM/F12 medium containing 2 mM L-glutamine, 400 μM G418, and 10% FBS. The APPswe cells were randomly divided into an experimental group and a control group, where the experimental group was treated with 300 μM copper ions, and the control group was not treated in any way. The experimental groups were randomly divided into an experimental group 1, an experimental group 2, an experimental group 3, and an experimental group 4, and the control group was divided into a control group 1, a control group 2, a control group 3, and a control group 4. The experimental group 1 and the control group 1 were both transiently transfected with 100 nM of a negative control (NC: SEQ ID NO: 9, 5′-UUGUACUACACAAAAGUACUG-3′) using liposome transfection; the experimental group 2 and the control group 2 were both transiently transfected with 100 nM of a novel miR-32533 mimics (SEQ ID NO: 10, 5′-UCUCUUCUGCUCUGUGUCACAGC-3′) using liposome transfection; the experimental group 3 and the control group 3 were both transiently transfected with 100 nM of a negative control (NCI: SEQ ID NO: 11, 5′-CAGUACUUUUGUGUAGUACAA-3′) using liposome transfection; the experimental group 4 and the control group 4 were both transiently transfected with 100 nM of a novel miR-32533 inhibitor (SEQ ID NO: 12, 5′-GCUGUGACACAGAGCAGAAGAGA-3′). The cells treated in the experimental and control groups were incubated at 37° C. for 24 h.

### 2. Detection of Cell Viability

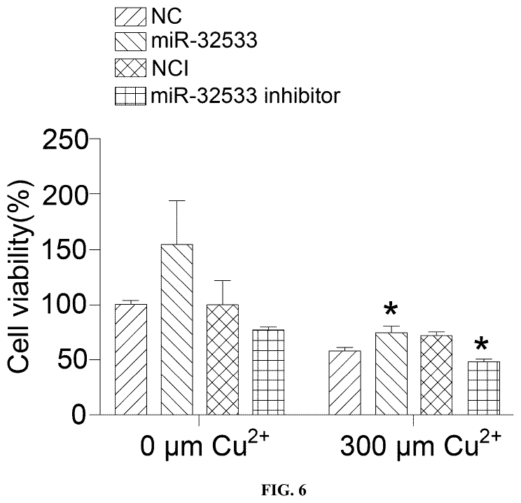

The cells treated with experimental groups 1 to 4 and control groups 1 to 4 in step 1 were taken, the cell supernatant was discarded, 90 μL of DMEM/F12 blank medium and 10 μL of MTS (10×) were added to each well, and incubated at 37° C. for 2 h. The OD value at a wavelength of 490 nm was detected by a multifunctional microplate reader, where the cell survival rate (%)=(OD490 experimental group-OD490 blank group)/(OD490 control group-OD490 blank group)×100%. The results were shown in FIG. 6, where the results were expressed as mean±SEM (n=3), and * indicated P<0.05 compared with the control group of transfections with irrelevant sequences (NC/NCI).

As shown in FIG. 6, miR-32533 mimics and miR-32533 inhibitor were transfected into APPswe cells to construct in vitro AD cell models of miR-32533 overexpression and knockdown, respectively. In APPswe cells treated with 300 μM copper ions, compared with the control group transfected with negative sequence control (NC/NCI), the overexpression of miR-32533 significantly increased the cell viability of APPswe cells, while the silencing of miR-32533 significantly reduced the survival rate of APPswe cells.

### 3. Flow Cytometry Detection

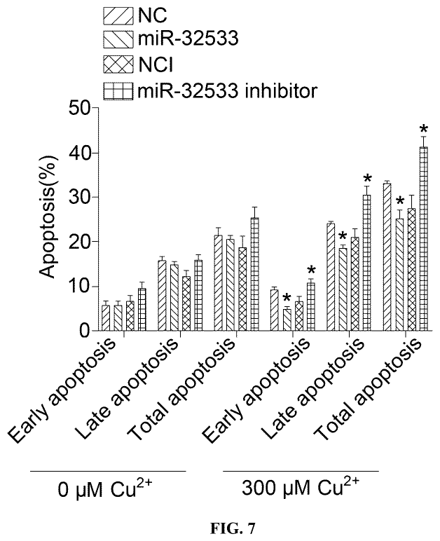

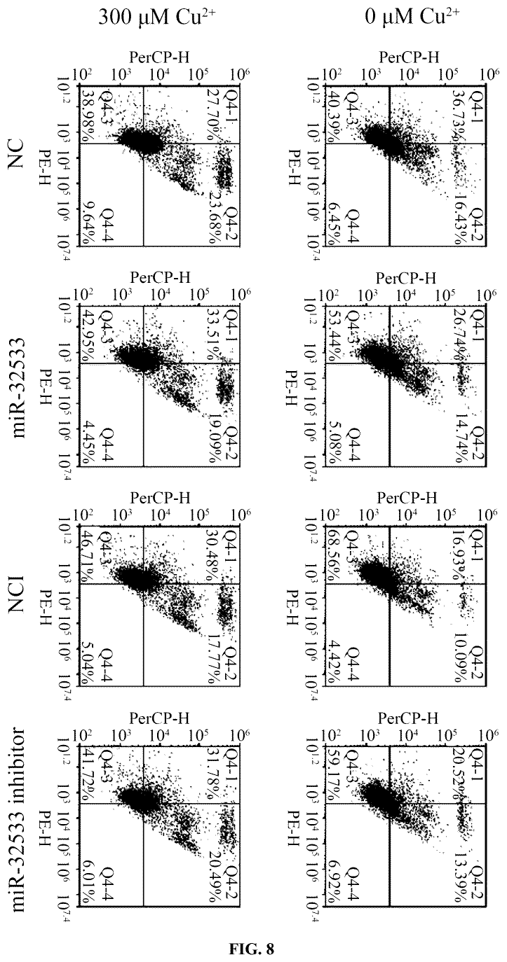

An Annexin V-PE/7-AAD apoptosis detection kit from Nanjing Vazyme Biotech was used, and the specific experimental operations were as follows:
(1) Cell collection: the cells treated with experimental groups 1 to 4 and control groups 1 to 4 in step 1 were digested with EDTA-free trypsin, centrifuged at 300 g and 4° C. for 5 min, and the supernatant was discarded.
(2) Washing cells: the cells were washed twice with pre-cooled PBS, centrifuged each time of washing at 300 g and 4° C. for 5 min, and the supernatant was discarded.
(3) Cell resuspension: 100 μL of 1× Binding Buffer was added and gently blown evenly until a single-cell suspension was obtained.
(4) Cell staining: 5 μL of Annexin V-PE and 5 μL of 7-AAD staining solution were added, incubated at room temperature for 10 min in the dark; and 400 μL of 1× Binding Buffer was added and mixed well gently.
(5) Detection: samples obtained in step (4) were detected using a flow cytometer (BD Biosciences, Franklin Lakes, NJ, USA) within 1 h. The detection results were shown in FIG. 7 to FIG. 8, where the results were expressed as mean±SEM (n=3), and * indicated P<0.05 compared with the control group transfected with negative sequence control (NC/NCI).

According to FIG. 7 to FIG. 8, overexpression of miR-32533 significantly reduced the early apoptosis rate, late apoptosis rate, and total apoptosis rate of APPswe cells. In contrast, silencing of miR-32533 exhibited the opposite effect, namely increasing the apoptosis rate of APPswe cells.

4. Western blot
4.1 Protein extraction
4.2 Protein quantification and denaturation
4.3 Western blot experiment
(1) Preparation of SDS-polyacrylamide gel was conducted.
(2) Protein loading and electrophoresis: 20 μg to 60 μg of protein was loaded into each well. The electrophoresis was in a constant voltage mode, at 80 V for 120 min, at 80 V for 60 min, or at 100 V for 40 min; when bromofen blue in the protein sample approached the bottom of running gel, the electrophoresis was terminated.
(3) Transfer (wet transfer): a PVDF membrane was activated in anhydrous methanol for 1 min, immersed in ultrapure water for 5 min, and equilibrated in transfer buffer for 10 min. A running gel containing protein samples was equilibrated in a transfer buffer for 10 min. A cathode-to-anode sandwich (successively sponge-filter paper-running gel-PVDF membrane-filter paper-sponge) was made with air bubbles removed, and transferred on ice in the constant current mode at 390 mA for 70 min.
(4) Blocking: the PVDF membrane was blocked in 5% skim milk at room temperature for 2 h to 3 h.
(5) Primary antibody incubation: a target antibody was prepared in a ratio of 1:1000 using 5% skim milk or 5% BSA, the membrane was cut according to a molecular weight of the target protein and the protein Marker of a corresponding molecular weight, and incubated at 4° C. with shaking overnight.
6. Membrane washing and secondary antibody incubation: the membrane was washed 5 times with Tris-buffered saline and Tween-20 (TBST) containing 0.1% Tween-20, with shaking for 6 min each time. A horseradish peroxidase (HRP)-conjugated secondary antibody of the corresponding species of the primary antibody was prepared and incubated at room temperature for 1 h; the membrane was washed 5 times with TBST, 6 min each time, to remove non-specific binding.
(7) Exposure imaging: an ECL luminescent liquid was added to the PVDF membrane and reacted for 3 min to 5 min, and then exposed on a Bio-Rad gel imager to obtain grayscale bands. Gray values of the bands were analyzed to calculate the relative expression levels of proteins.

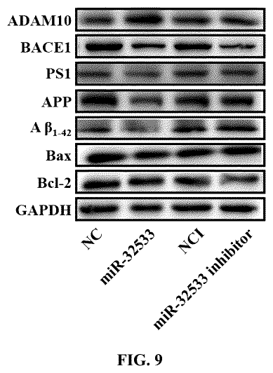

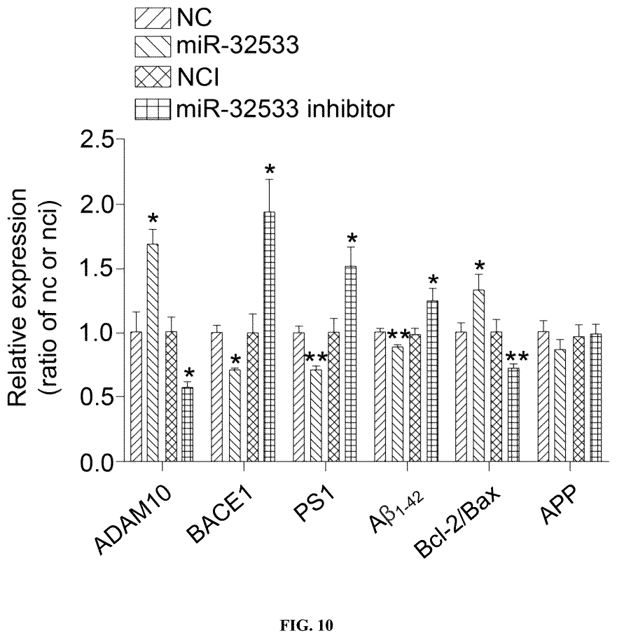

According to the basic operation of Western blot, changes in the levels of related molecular proteins were detected. During the primary antibody incubation, 5% skim milk or 5% BSA was used to prepare the target antibodies (ADAM10, BACE1, PS1, APP, Aβ1-42, Bcl-2 and Bax) at a ratio of 1:1000. During the secondary antibody incubation, HRP-coupled secondary antibodies of the corresponding species of the primary antibody were prepared. Finally, the exposure imaging was conducted: the ECL luminescent liquid was added to the PVDF membrane and reacted for 3 min to 5 min, and then exposed on a Bio-Rad gel imager to obtain grayscale bands. Gray values of the bands were analyzed to calculate the relative expression levels of proteins. The results were shown in FIG. 9 and FIG. 10, where the results were expressed as mean±SEM (n=3), * indicated P<0.05 compared with the control group transfected with negative sequence controls (NC/NCI), and ** indicated P<0.01 compared with the control group transfected with negative sequence controls (NC/NCI).

FIG. 9 and FIG. 10 showed that the silencing of miR-32533 reduced the ratio of anti-apoptotic and apoptotic markers (Bcl-2/Bax), and the up-regulation of miR-32533 expression significantly inhibited the expression level and activity of endogenous apoptotic signaling pathway markers. The overexpression of miR-32533 significantly reduced the expression level of Aβ1-42, promoted the expression level of ADAM10 protein in the non-amyloid metabolic pathway of Aβ, and inhibited the expression levels of BACE1 and PS1 in the amyloid metabolic pathway of Aβ. The silencing of miR-32533 significantly reduced the expression level of ADAM10, promoted the expression levels of BACE1 and PS1, and thus promoted the excessive generation of Aβ 1-42. This suggested that miR-32533 was involved in the pathological process of Aβ generation and in AD-related neuronal damage. Therefore, the overexpression of miR-32533 could significantly improve the cell viability of APPswe cells and inhibit apoptosis-like degeneration, thereby exerting neuroprotective effects.

### EXAMPLE 4

### Expression Changes of the miR-32533 in Plasma of AD Patients

1. RT-PCR detection

Blood was collected from 11 AD patients and 10 healthy adult volunteers (HAVs) of the same age as the experimental materials. The expression level of miR-32533 in the plasma of AD patients and HAVs of the same age was detected by RT-PCR, the detection steps are as follows:

1.1 Extraction of miRNA From Plasma Samples

The plasma RNA extraction kit (miRNeasy Serum) from QIAGEN, Germany was selected, specifically including:
(1) Plasma sample collection: the blood was collected from AD patients and HAVs using anticoagulant tubes, centrifuged at 1,000 g for 15 min to obtain the upper plasma layer, and 200 μL of plasma was dispensed into each tube and frozen in a-80° C. refrigerator.
(2) 200 μL of serum was taken and added with 1 mL of QIAzol Lysis Reagent, mixed well by inversion, and allowed to stand at room temperature for 5 min.
(3) 200 μL of chloroform was added, shaken vigorously for 30 s, allowed to stand at room temperature for 3 min, and centrifuged at 12,000 g and 4° C. for 15 min.
(4) An upper aqueous phase was aspirated and added into 1.5-fold volume of anhydrous ethanol, and shaken to mix well.
(5) A mixture of the upper aqueous phase and anhydrous ethanol was pipetted into an RNeasy MinElute spin column, centrifuged at 8,000 g for 30 s, and a filtrate was discarded to collect RNA.
(6) 700 μL of RWT buffer was added, centrifuged at 8,000 g for 30 s, and the filtrate was discarded.
(7) 500 μL of RPE buffer was added, centrifuged at 8,000 g for 30 s, and the filtrate was discarded.
(8) 500 μL of 80% ethanol was added, centrifuged at 8,000 g for 2 min, and the filtrate was discarded.
(9) The RNA collection column was replaced in the collection tube, centrifuged at 8,000 g for 5 min, and the filtrate and collection tube were discarded.
(10) The RNA collection column was transferred to a new 1.5 mL centrifuge tube, 14 μL of RNase-free water was added, and the tube was centrifuged at 8,000 g for 1 min to elute and collect the RNA.
(11) The concentration and purity of RNA were measured on a Spark 20M multifunctional microplate reader and the RNA was stored at −80° C. for subsequent experiments. 1.2 RT-PCR of miRNA

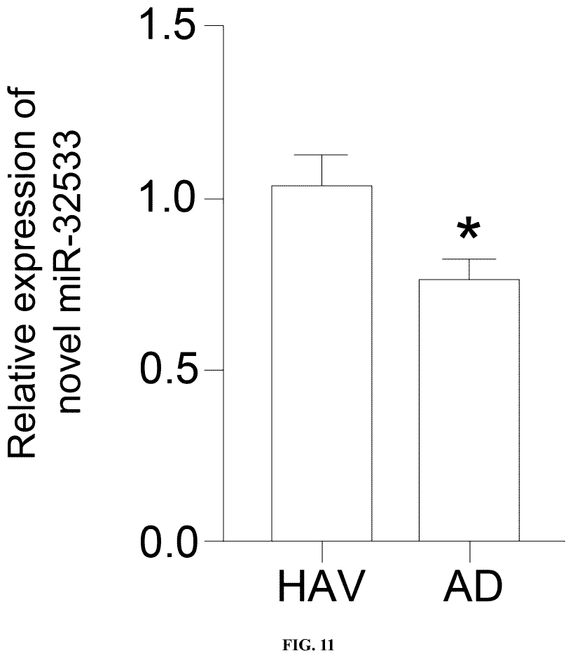

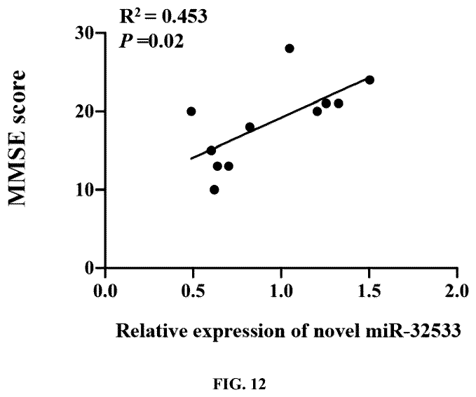

The relative expression level of miR-32533 in the plasma of AD patients and HAVs of the same age was detected using step 1.2 of Example 2. The results were shown in FIG. 11 to FIG. 12, where the results were expressed as mean±SEM (n=3), and * indicated P<0.05 compared with the plasma of HAVs of the same age.

As shown in FIG. 11 FIG. 12 and FIG. 3, it was evidenced that miR-32533 was significantly downregulated in the plasma of AD patients and was significantly positively correlated with the MMSE score of AD patients (R2-0.453, P<0.05). MMSE score results were used to evaluate the cognitive function of patients based on the Mini-Mental State Examination (MMSE) scale for AD. The total score of the MMSE scale was 30, of which 0-9 represented severe cognitive impairment, 10-20 represented moderate cognitive impairment, 21-26 represented mild cognitive impairment, and greater than 27 represented normal cognitive function.

### 2. ROC Curve Detection

The sample values were back-substituted with the expression level of miR-32533 as an independent variable, and AD patients and HAV controls were used as dependent variables.

The ROC curves of each index were fitted with sensitivity as an ordinate (true positive rate) and 1-specificity as an abscissa (false positive rate). The value of biomarkers was determined in plasma for the independent diagnosis and combined diagnosis.

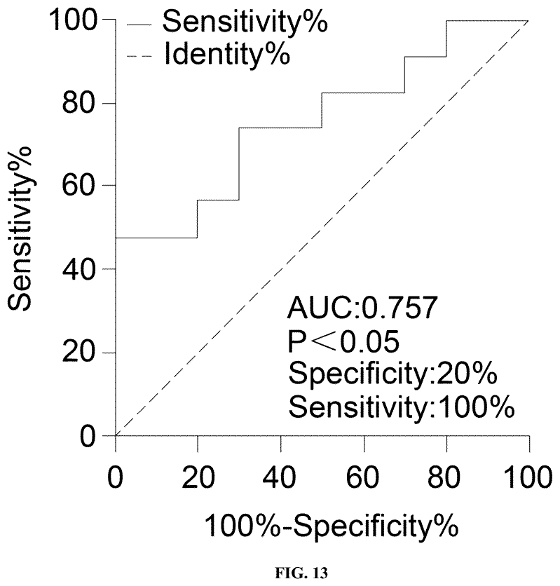

The ROC curve was measured using step 2 of Example 2, and the results were shown in FIG. 13. It could be seen that miR-32533 exhibited desirable diagnostic value as a biomarker in the plasma of AD patients and HAV controls (AUC=0.757, Sensitivity=100%, Specificity=20%). Combined with FIG. 11 to FIG. 12, the miR-32533 was significantly downregulated during the pathological process of AD and had a desirable clinical diagnostic value in the plasma of AD patients.

### EXAMPLE 5

### Prediction for Target Genes of miR-32533

1. The target gene analysis of miR-32533 was conducted using the bioinformatics software TargetScan (//www.targetscan.org/), miRanda (//www.microrna.org/microrna/home.do), and TarBase (//carolina.imis.athena-innovation.gr/diana_tools/web/index.php?r=tarbasev8%2Findex), and it was found that CREB5 was associated with AD. Specifically:

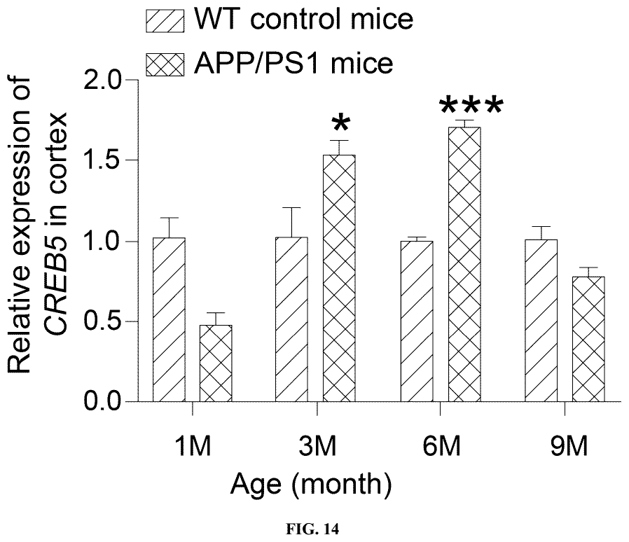

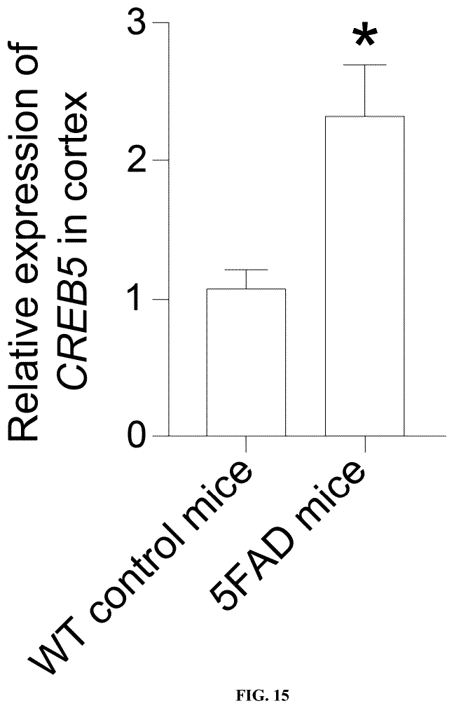

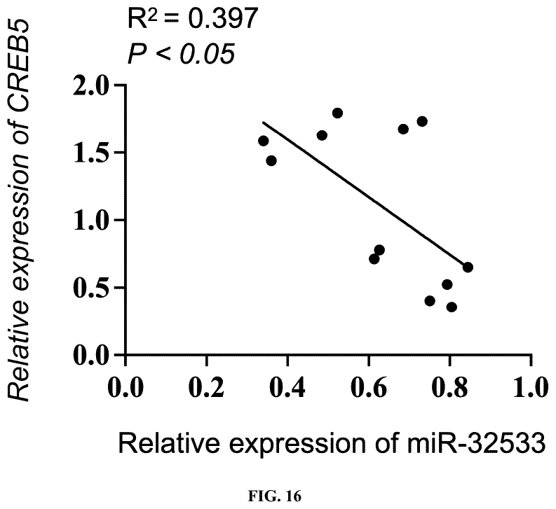

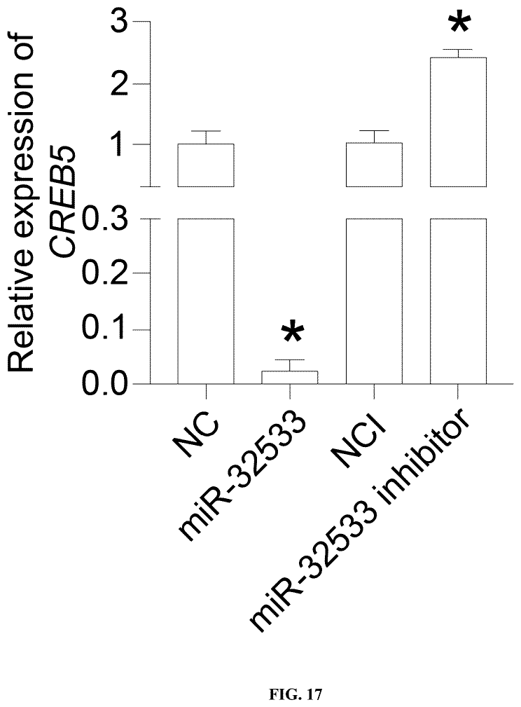

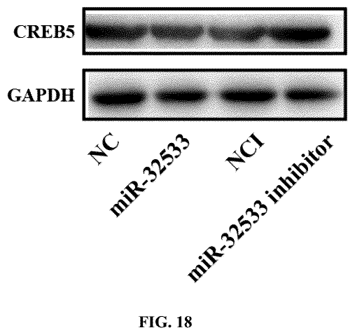

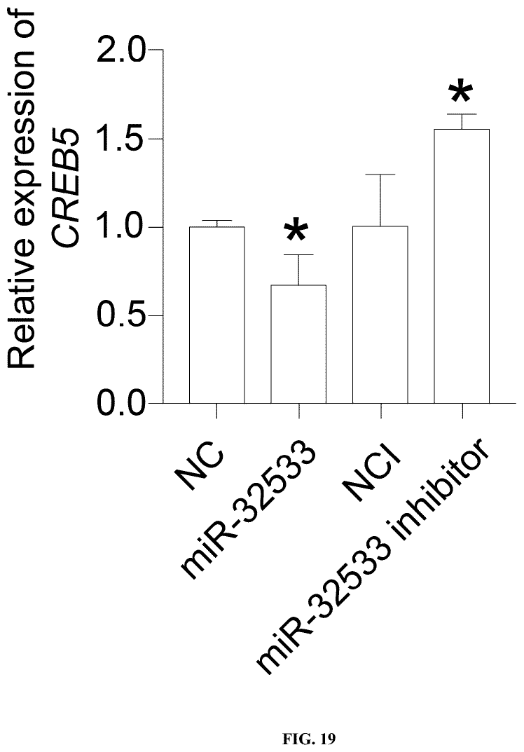

The qRT-PCR detection revealed that the CREB5 was significantly upregulated in the cerebral cortex of APP/PS1 mice (FIG. 14, where the results were calculated as mean±SEM (n=3), * indicated P<0.05 in APP/PS1 mice compared with WT control mice, *** indicated P<0.001 in APP/PS1 mice compared with WT control mice; M represented month). The target gene was significantly upregulated in the cerebral cortex of 5×FAD mice (FIG. 15, where the results were calculated as mean±SEM (n=3), * indicated P<0.05 in 5×FAD mice compared with WT control mice), and the expression levels of miR-32533 and CREB5 were significantly negatively correlated (FIG. 16, R2-0.397, P<0.05). Meanwhile, miR-32533 could significantly reduce the expression of CREB5 at the mRNA and protein levels, while silencing of miR-32533 could significantly promote the expression levels of CREB5 mRNA and protein (FIG. 17 to FIG. 19, where the results were expressed as mean±SEM (n=3), * indicated P<0.05 compared with the control group transfected with an negative sequence control (NC/NCI)).

### 2. Construction of Dual Luciferase Reporter Gene and Detection Steps:

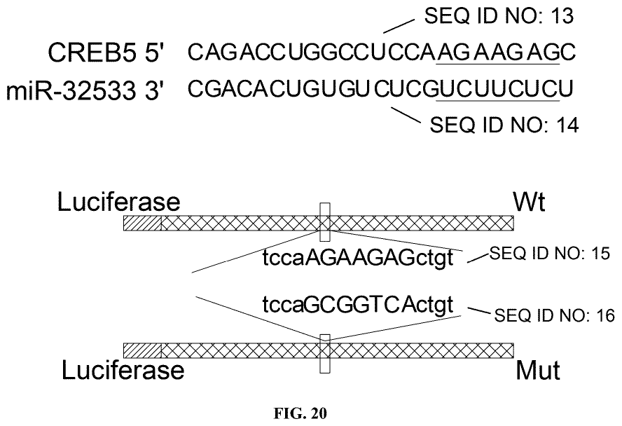

The DNA sequence matching between miR-32533 and the 3′UTR of CREB5 was predicted by the bioinformatics software Starbase, miRbase, and Targetscan. Primer 5.0 was used to design specific primers for PCR amplification to obtain a DNA fragment, which was then inserted into a pmirGLO dual luciferase reporter system plasmid (Promega) to construct a pmirGLO-CREB5-3′UTR-WT plasmid (referred to as WT). The miR-32533 and CREB5 binding sites were mutated to construct a pmirGLO-CREB5-3′UTR-MUT plasmid (referred to as MUT), and the results were shown in FIG. 20. The pmirGLO-CREB5-3′UTR-WT (pmirGLO-CREB5-3′UTR-MUT) reporter vector and miR-32533 were transfected into HEK293T cells by liposome transfection, while the pmirGLO-CREB5-3′UTR-WT (pmirGLO-CREB5-3′UTR-MUT) reporter vector and NC were transfected into HEK293T cells as a control. 48 h after transfection, the activities of firefly and Renilla luciferases were detected using the dual-color luciferase reporter assay system (Promega) to obtain a standardized relative reporter activity.

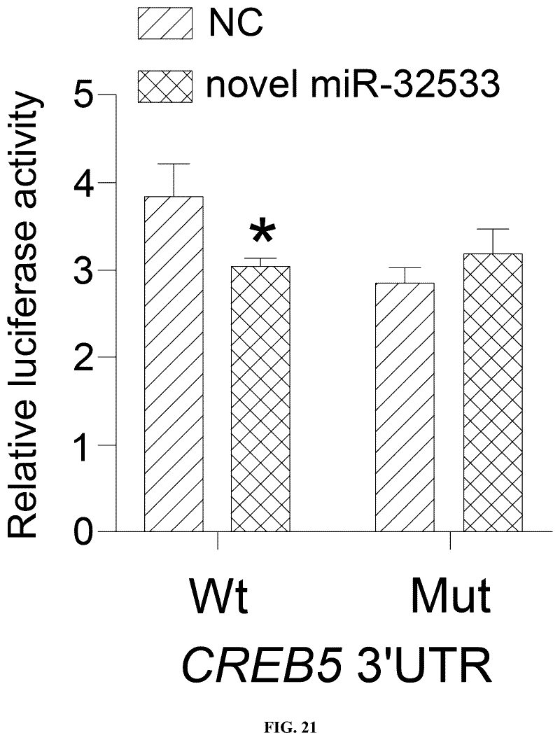

The dual luciferase reporter gene was detected using the Dual Luciferase Reporter Assay Kit from Nanjing Vazyme Biotech Co., Ltd., and the specific experimental operations were as follows:
(1) Cell lysis: after treatment, the HEK293 cells were washed twice with PBS, and 100 μL of 1× cell lysis buffer (5× Cell Lysis Buffer was diluted to 1× Cell Lysis Buffer with deionized water) was added to allow lysis on a shaker for 15 min; after centrifugation at 12,000 g for 2 min, the cell supernatant was taken for detection.
(2) Detection of Firefly luciferase activity: 20 μL of cell lysate supernatant and 100 μL of luciferase substrate were added to a 96-well white plate, and the fluorescence was immediately detected using a Spark 20M multifunctional microplate reader and recorded as Firefly.
(3) Detection of Renilla luciferase activity: 100 μL of a prepared Renilla working solution was added to the reaction working solution in step (2) to detect the fluorescence value of Renilla luciferase, recorded as Renilla.
(4) Analysis: a ratio of Firefly/Renilla was calculated as a relative fluorescence value, and the results were shown in FIG. 21. The results in FIG. 21 were calculated as mean±SEM (n=3), where Wt group * indicated that P<0.05 compared with the control group transfected with the irrelevant sequence NC; Wt represented the relative fluorescence value after co-transfection of miR-32533 (referred to as novel miR-32533) and pmirGLO-CREB5-3′UTR-WT, namely the results of the dual luciferase reporter gene assay; Mut represented the relative fluorescence value after co-transfection of miR-32533 (referred to as novel miR-32533) and pmirGLO-CREB5-3′UTR-MUT, namely the results of the dual luciferase reporter gene assay.

According to FIG. 21, it could be seen that co-transfection of miR-32533 and pmirGLO-CREB5-3′UTR-WT significantly reduced the luciferase activity in HEK293 cells; however, co-transfection of miR-32533 and pmirGLO-CREB5-3′UTR-MUT did not result in significant changes. This indicated that the miR-32533 directly bound specifically to CREB5 and specifically negatively regulated the expression level of CREB5 at the transcriptional and translational levels.

According to the above examples, the miR-32533 provided by the present disclosure can be used as a biomarker for detecting AD, can specifically bind to CREB5, reduce the expression level of CREB5, inhibit the generation of Aβ, thereby effectively preventing and treating AD.

Although the above examples provide a detailed description of the present disclosure, they are only a part of, not all of, the examples of the present disclosure. Other examples may also be obtained by those skilled in the art based on the example without creative efforts, and all of these examples shall fall within the protection scope of the present disclosure.

What is claimed is:

1. A biomarker miR-32533 for Alzheimer's disease, wherein the nucleotide sequence of the biomarker miR-32533 is set forth in SEQ ID NO: 1.

2. Use of the biomarker miR-32533 according to claim 1 in preparation of a drug for treating Alzheimer's disease.

3. The use according to claim 2, wherein the biomarker miR-32533 has one or more functions selected from the group consisting of I) to VI):
I) increasing an expression level of ADAM10 in a non-amyloid metabolic pathway of β-amyloid (Aβ);
II) reducing expression levels of BACE1 and PS1 in an amyloid metabolic pathway of the Aβ;
III) inhibiting generation of the Aβ;
IV) exerting a neuroprotective effect;
V) increasing a cell viability and reducing apoptosis; and
VI) reducing an expression level of CREB5.

4. A drug for treating Alzheimer's disease, wherein active ingredients of the drug comprise miR-32533; and wherein
the nucleotide sequence of miR-32533 is set forth in SEQ ID NO: 1.

5. Use of the miR-32533 according to claim 1 in preparation of a kit being one or more selected from the group consisting of a) to d):
a) a kit for screening a population with Alzheimer's disease;
b) a kit for diagnosing the population with Alzheimer's disease;
c) a kit for monitoring a treatment status of the population with Alzheimer's disease; and
d) a kit for monitoring prognosis of the population with Alzheimer's disease.

6. The kit according to claim 5, wherein the kit comprises a reagent for detecting the miR-32533.


SEQUENCE LISTING (16 sequences)

SEQ ID NO: 1 - RNA, 23 nt, synthetic construct
  source 1..23: mol_type = other RNA
tctcttctgctctgtgtcacagc

SEQ ID NO: 2 - RNA, 50 nt, synthetic construct
  source 1..50: mol_type = other RNA
tctcttctgctctgtgtcacagcaacagaaagctgcactgctgaggaaca

SEQ ID NO: 3 - DNA, 50 bp, synthetic construct
  source 1..50: mol_type = other DNA
gtcgtatccagtgcagggtccgaggtattcgcactggatacgacgctgtg

SEQ ID NO: 4 - DNA, 21 bp, synthetic construct
  source 1..21: mol_type = other DNA
cgcgtctcttctgctctgtgt

SEQ ID NO: 5 - DNA, 20 bp, synthetic construct
  source 1..20: mol_type = other DNA
agtgcagggtccgaggtatt

SEQ ID NO: 6 - DNA, 50 bp, synthetic construct
  source 1..50: mol_type = other DNA
gtcgtatccagtgcagggtccgaggtattcgcactggatacgacaaaata

SEQ ID NO: 7 - DNA, 20 bp, synthetic construct
  source 1..20: mol_type = other DNA
caaattcgtgaagcgttcca

SEQ ID NO: 8 - DNA, 20 bp, synthetic construct
  source 1..20: mol_type = other DNA
agtgcagggtccgaggtatt

SEQ ID NO: 9 - RNA, 21 nt, synthetic construct
  source 1..21: mol_type = other RNA
ttgtactacacaaaagtactg

SEQ ID NO: 10 - RNA, 23 nt, synthetic construct
  source 1..23: mol_type = other RNA
tctcttctgctctgtgtcacagc

SEQ ID NO: 11 - RNA, 21 nt, synthetic construct
  source 1..21: mol_type = other RNA
cagtacttttgtgtagtacaa

SEQ ID NO: 12 - RNA, 23 nt, synthetic construct
  source 1..23: mol_type = other RNA
gctgtgacacagagcagaagaga

SEQ ID NO: 13 - RNA, 23 nt, synthetic construct
  source 1..23: mol_type = other RNA; note = fragment of CREB5
cagacctggcctccaagaagagc

SEQ ID NO: 14 - RNA, 23 nt, synthetic construct
  source 1..23: mol_type = other RNA; note = fragment of miR-32533
tctcttctgctctgtgtcacagc

SEQ ID NO: 15 - DNA, 15 bp, synthetic construct
  source 1..15: mol_type = other DNA; note = inserted DNA fragment
tccaagaagagctgt

SEQ ID NO: 16 - DNA, 15 bp, synthetic construct
  source 1..15: mol_type = other DNA; note = mutated DNA fragment
tccagcggtcactgt